(19) United States
(12) Patent Application Publication
(10) Pub. No.: US 2020/0265920 A1
(43) Pub. Date: Aug. 20, 2020

(54) A SYSTEM FOR DETERMINING DIPLOTYPES

(71) Applicants: The Children's Mercy Hospital, Kansas City, MO (US); The Curators of the University of Missouri, Columbia, MO (US)

(72) Inventors: Greyson Twist, Prairie Village, KS (US); Neil Miller, Santa Fe, NM (US); Deendayal Dinakarpandian, Prairie Village, KS (US)

(21) Appl. No.: 16/067,908

(22) PCT Filed: Jan. 7, 2017
(86) PCT No.: PCT/US17/12647

§ 371 Date: Jul. 3, 2018

Related U.S. Application Data
(60) Provisional application No. 62/275,975, filed on Jan. 7, 2016.
(60) Provisional application No. 62/288,271, filed on Jan. 28, 2016.

(51) Int. Cl. G16B 20/00; C12Q 1/6869; G16B 30/00; G16B 5/00; G16H 50/20

(52) U.S. Cl. CPC G16B 20/00; C12Q 1/6869; C12Q 2537/165; G16B 5/00; G16H 50/20; G16B 30/00

(57) ABSTRACT

A system is provided for predicting the diplotype of an individual comprising the steps of (a) initializing a data store with a plurality of pre-defined locus positions and a plurality of pre¬defined nomenclatures, (b) retrieving genomic sequencing results of an individual, (c) comparing a plurality of variant calls and associated zygosities with the plurality of pre-defined locus positions and plurality of pre-defined nomenclatures to identify the individual's diplotype, (d) assigning a score to each of the plurality of pre-defined locus positions based on the comparison of step (c), (e) reporting at least one score (typically the highest score) and associated diplotype to an end user. The present invention can further comprise the step of using the associated diplotype of step (e) to predict the biological impact or phenotype of the individual.

### BACKGROUND ART

A protein's function is directly determined by the genomic sequence which encodes it. Using a common, but arbitrary, genomic sequence coding for the specific protein a “reference” genomic sequence can be defined. This allows for cataloging of genomic variations as compared to the reference. Variants observed together in a single sequence are designated as a haplotype, when many haplotypes are observed across a single gene locus these haplotypes can be individually named or labeled to four a gene nomenclature. For gene haplotype/nomenclature sets additional information can be associated with the label to expand on how the variations in the sequence will impact the protein functions, for example: gain or loss of function in comparison to the “reference” sequence, alteration of allosteric regulation sites, gain or loss of protein-protein interaction domains, gain or loss of catalytic reaction sites, increase or decrease in substrate transportation potential.

The haplotype/nomenclature framework can also be defined for any genomic regions identified by positional start and stop and containing variation (be it sequential or chemical modification), and in which intra haplotype variation impacts biological activity in some way. Haplotype definitions are not limited to protein encoding regions, for example with genomic regions that act to regulate protein production but are not actually transcribed. This regulation could be via transcription enhancer binding, DNA methylation, or sequence variation affecting histone binding.

Although some haplotyping assays exist, they are difficult and time consuming making them prohibitive to run. For example, determining medication dose and predicting medication side effects from genomic information is time consuming, complex, and prone to human error. A critical step in this process is to determine the contributing alleles from specific gene loci that impact an individual drug. These can be used to determine the relative activity for a single person and how they may react to a drug. The anticoagulant Warfarin is often pointed to as a success story for pharmacogenomics. Genotyping prior to dosing, or genotyping while giving trial doses at sub-clinical levels can indicate the potential for adverse drug reactions. In the case of Warfarin, testing two genes, CYP2C9 and VKORC1, resolves roughly 40% of the variation seen but leaves unresolved the other 60% of dose variations. Currently most genotyping platforms only look at a single variation, or a small set of variations which are then used to attempt locus phenotype prediction. These existing methods are limited to decision trees, are reliant on a single technological platform for output, and miss rare or novel variants that may also impact haplotype.

There exists the need to deliver individualized haplotype information based on data sets that relate to a single person. Further, it would be beneficial to have a system that is platform independent and uses pre-defined locus positions and nomenclatures that match the reference human genome to allow for identification of the individual's diplotype and when possible, a predicted biological impact.

### DISCLOSURE OF INVENTION

The system of the present invention is a computational method for automated derivation of diploid functional haplotypes from genomic sequence information, which can be from phased or unphased genomic sequence information. A system is provided for predicting the diplotype of an individual comprising the steps of (a) initializing a data store with a plurality of pre-defined locus positions and a plurality of pre-defined nomenclatures, (b) retrieving genomic sequencing results of an individual, (c) comparing a plurality of variant calls and associated zygosities with the plurality of pre-defined locus positions and plurality of pre-defined nomenclatures to identify the individual's diplotype, (d) assigning a score to each of the plurality of pre-defined locus positions based on the comparison of step (c), (e) reporting at least one score (typically the highest score) and associated diplotype to an end user. In another embodiment of the invention, the present invention can further comprise the step of using the associated diplotype of step (e) to predict the biological impact or phenotype of the individual.

In another embodiment of the invention, the system of the present invention uses whole genome sequencing (WGS) or any similar method for retrieving genomic information as an electronic decision support system to aid a physician or other medical care provider to inform drug choice and dosing for a patient. To achieve this, the system of the present invention uses automated identification of genomic variation in genes involved in drug absorption, distribution, metabolism, excretion and response (ADMER). Cytochrome P450 family 2, subfamily D, polypeptide 6, (CYP2D6), is one of the most important enzymes of bioactivation or elimination of endogenous and exogenous biochemicals. CYP2D6 activity is predominantly governed by genomic variation; however it is technically arduous to haplotype. The nucleotide sequence of CYP2D6 is highly polymorphic, but the locus also participates in diverse structural variations, including gene deletion, duplication, multiplication events and rearrangements with the nonfunctional, neighboring CYP2D7 and CYP2D8 genes.

The system of the present invention comprises (1) a probabilistic scoring system, and (2) an enabling automated ascertainment of CYP2D6 activity scores from genomic data. When compared with reference methods (manual evaluation of diverse genotyping assays including copy number, variation determination, long-range PCR analysis and Sanger sequencing), the system of the present invention had an analytic sensitivity of 97% (59 of 61 diplotypes) and analytic specificity of 89% (105 of 118 haplotypes), which was greater than that of Sanger sequencing or TaqMan® (a registered trademark of Thermo Fisher Scientific, Inc.) genotyping (86% and 83% specificity, respectively). The clinical sensitivity of the system of the present invention was 94%, and clinical specificity was 98% (57 of 58 activity scores). The system of the present invention is extensible to functional variation in all ADMER genes, and may be performed at marginal incremental financial and computational costs in the setting of diagnostic WGS.

Other and further objects of the invention, together with the features of novelty appurtenant thereto, will appear in the course of the following description.

### Definitions

Haplotype is a specific allele that progeny inherited from one parent.

Diplotype is a specific combination of two haplotypes.

Phenotype is the composite of an organism's observable characteristics or traits, such as its morphology, development, biochemical or physiological properties, phenology, behavior, and products of behavior. A phenotype results from the expression of an organism's genes as well as the influence of environmental factors and the interactions between the two. When two or more clearly different phenotypes exist in the same population of a species, the species is called polymorphic.

Data store refers to any computer readable format which can retain information about haplotype labels and defining variant sets, including but not limited to ordered file systems, referential data stores, or NoSQL style database.

Next generation or NextGen sequencing refers to high-throughput sequencing methods which can interrogate genetic loci at random or in a targeted manner. These technologies include, but are not limited to, Illumina (Solexa) sequencing by Illumina, Inc., Roche 454 by Roche Diagnostics, Ion Torrent by Thermo Fisher Scientific Inc., SOLiD by Thermo Fisher Scientific Inc., Pac Bio by Pacific Biosciences of California, Inc., and Nanopore by Oxford Nanopore Technologies.

### BRIEF DESCRIPTION OF THE FIGURES

FIG. 1 is a depiction of the structure of the highly polymorphic CYP2D6/2D7/2D8 locus, showing the relative activity of CYP2D6 for the reference and 13 variant haplotypes.

FIG. 2 is a depiction of long-range PCR products used to define CYP2D7/2D6 hybrid genes.

FIG. 3 is a depiction of in silico modeling of the uniqueness of alignments of simulated short-read sequences to the region of Chromosome 22 containing CYP2D6, CYP2D7, and CYP2D8.

FIG. 4 is a depiction of in silico modeling of the uniqueness of alignments of simulated short-read sequences to the region of Chromosome 22 containing CYP2D6 and CYP2D7.

FIG. 5 is a panel of genotyping assays interrogating selected more common SNPs.

FIG. 6 is a block diagram showing a computer system of the present invention.

### BEST MODE FOR CARRYING OUT THE INVENTION

The system of the present invention can be applied to complex diseases where actionable clinical results have been difficult to derive from whole genome sequences. Despite abundant knowledge of genomic variants conferring risk, pathogenicity probability is often related to single nucleotide variation. By extending the system of the present invention from the integration of intra-locus variation to include multiple loci, a cumulative risk score for complex diseases in individual patients can be calculated. Use of such methods to genome-wide association datasets allows parameterization of the scoring algorithm for individual common diseases.

Some portions of the detailed descriptions which follow are or may be presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, terms such as “processing” or “computing” or “calculating” or “determining” or “displaying” or the like, refer to the action and processes of a computer system, or similar computing device, that manipulates and transforms data represented as physical (e.g., electronic) quantities within the computer system's registers and memories into other data similarly represented as physical quantities within the computer system memories or registers or other such information storage, transmission or display devices.

The system of the present invention is a computational method for automated derivation of diploid functional haplotypes from whole genome sequencing (WGS) or any other method now known or that is known in the future that delivers genomic information, including for example, whole genome DNA sequences, RNA sequences, methylation sequences, array based hybridization. The system of the present invention is a computational method for automated derivation of diploid functional haplotypes from genomic sequence information. A system is provided for predicting the diplotype of an individual comprising the steps of (a) initializing a data store with a plurality of pre-defined locus positions and a plurality of pre-defined nomenclatures, (b) retrieving genomic sequencing results of an individual, (c) comparing a plurality of variant calls and associated zygosities with the plurality of pre-defined locus positions and plurality of pre-defined nomenclatures to identify the individual's diplotype, (d) assigning a score to each of the plurality of pre-defined locus positions based on the comparison of step (c), (e) reporting at least one score (typically the highest score) and associated diplotype to an end user. In another embodiment of the invention, the present invention can further comprise the step of using the associated diplotype of step (e) to predict the biological impact or phenotype of the individual.

The system of the present invention is extensible to any polymorphic locus in which a comprehensive library of functionally relevant haplotypes and defining variant sets can be determined, and for which paired short reads align unambiguously. An example would be the Human Leukocyte Antigen (HLA) regions, where these proteins encode for cell surface markers critical to regulation of the immune system. The HLA haplotypes available to the immune system are critical in a variety of health settings including but not limited to, transplant setting requiring proper matching between the HLA classes to ensure that the host's immune system will not attack the graft and vice versa. In autoimmune disease HLA type DR4 is associated with autoimmune disorders Rheumatoid arthritis and Diabetes Mellitus type 1 while having HLA type DQ2 and DQ8 are associated with Celiac disease.

The system of the present invention provides an algorithm to impute diplotypes from genomic sequence data. The algorithm is a probabilistic scoring system that computes the score as the noise corrected likelihood that the sequence data matches a particular diplotype. The diplotype with the maximum likelihood is then assigned to the individual.

Step 1. For each possible diplotype, compute the noise corrected likelihood based on the observed variants.

Step 2. Then sort the diplotypes in descending order by score, and report the diplotype with the highest score as the most probable.

Such an algorithm is necessary because direct conversion of locus genotype sets to functional allelic, haplotype, sets is not possible since current genomic reporting methods are unphased and give no information regarding allele origin. The algorithm of the present invention is also useful for phased data by being a rapid, automated system for detecting haplotype sets, which can help to remove or minimize human error. Global or local sequence alignment algorithms fail because of noise due both to sequencing errors and variants that are not represented in known/defined alleles. The latter is particularly crucial since some allele definitions are based on SNPs in exonic regions rather than complete haplotype sequences. Furthermore, there are no rigorous scoring paths making it difficult to recognize the correct answer among the possible solutions. Thus, the problem is akin to de novo peptide sequencing from tandem mass spectrometry in the presence of false positives and false negatives.

A probabilistic scoring system determines the most likely diplotype match to the WGS-derived.vcf file (Vt) of a test sample, t, based on prior computation of all theoretical haplotypes and corresponding functional alleles (as defined by the Human P450 Nomenclature Committee). For the haplotypes of interest, a defining variant set is determined. The complete set of possible diplotypes is generated by combining the variant sets for each pair of haplotypes. For WGS of test sample t, the system of the present invention retrieves the position and zygosity of each variant in the .vcf file, Vt that is compared with each possible diplotype D1-D(n). For a diplotype Da and Vt, X variants are common, Y variants are in Vt only, and Z variants are found in the Da only [X=(Vt∩Da), Y=(Vt−Da), and Z=(Da−Vt)]. A variant location which is homozygous in Vt but heterozygous in the Da set will result in X+1 and Z+1 score adjustments.

To adjust for variant call errors, the scores are adjusted by the sensitivity (sens) and specificity (spec) of variant calling. Assuming independence of variant calls, the score for each variant is reported as a likelihood ratio. For instance, a reported variant (type X) that matched a candidate diplotype is scored as P(Predicted|Present)/P(Predicted|Absent)=Sensitivity/(1−Specificity), type Y scored as (Predicted|Absent)/P(Predicted/Present)=(1−Specificity)/Sensitivity, and type Z scored as P(Not Predicted|Present)/P(Not Predicted|Absent)=(1−Sensitivity)/Specificity. Thus, X was adjusted by A=[sens/(1−spec)], Y adjusted by B=[(1−sens)/spec], and Z adjusted by C=[(1−spec)/sens]. The overall score is the product of likelihood ratios of a diplotype sample set match [score=(A^{x})*(B^{y})*(C^{z})]. Resultant diplotypes were returned in a sorted list with the highest index, max(P), reported to the output file. The activity corresponding to the highest scoring diplotype was reported.

Data inputs for the system of the present invention were variant call format (.vcf) file, a gene directory with chromosomal position, and nomenclature file for each locus to be diplotyped. The position file contained the location of the gene transcript [Chr:start-stop] according to the Human genome GRCh37 reference. The nomenclature file contained the full set of known possible haplotypes, one per line, in the format [allele_name<tab>var1,var2,var3], with variants annotated as [Chr˜start˜stop˜var]. The output is the most likely diplotype for that sample. The system of the present invention was implemented in the Java programming language but other programming languages can be used.

“Variant detection” is a process by which differences between the individual and the reference genome, or “variants”, are identified. Variant calls will note a genomic position and the change observed in the individual, for example “chromosome 22, position 12345, reference is A variant is G” can also be notated as “chr22:12345 A>G”. Variant call format (VCF) is a standard file format for recording variants that includes positional information as well as zygosity of the variant call (e.g. heterozygous for the variant where one allele is the reference allele and one allele is the variant allele, or homozygous for the variant where both alleles are the variant allele). VCF compactly describes both variant and zygosity information. VCF is only one variant format, however, and the method of the present invention may use a different format.

To determine if possible copy number variation was present a BAM file (.bam) and a BED file (.bed) are used. The BAM file contains aligned reads against a reference genome and the BED file containing a list of sentinel regions marked by position against the aligned reference. Sentinel regions are evaluated for depth of coverage as are paired control regions. Significant deviation from expected ratios of coverage indicates the possible presence of copy number variation.

In order to demonstrate the utility of the present system, it was used to solve a difficult haplotype identification problem. Using the system to successfully deliver the haplotype results in the following situation, provides assurance that the system can work with less complicated examples. The Human Cytochrome P450 Allele Nomenclature data store annotates haplotype sets for CYP genes involved in drug metabolism. These allelic haplotypes define specific genomic variation in individuals that are associated to poor, intermediate, extensive and ultrarapid metabolizer phenotypes. Modern sequencing platforms produce individual variant calls with high sensitivity and specificity, but technical limitations (e.g. short read lengths) make the determination of haplotype difficult or impossible. Additionally, even in the presence of phased variant calls, identifying diplotypes manually is a time consuming and error prone process. The practical result of this is that there exists a gap between the ability of NextGen sequencing to produce high quality sequencing data rapidly and the ability of medical practitioners to make use of that data to inform drug dosing by leveraging the existing allelic haplotype data. Data may be stored on a file system disk, as a relational data system, or other known means of storing data. Data found in the data store may be entered manually or automatically loaded or populated.

The system of the present invention addresses this issue by using a probabilistic scoring system to identify a patient's combination of haplotypes, or diplotype, from a standard variant call file produced by NextGen sequencing workflows. The automation of this task reduces the translation of individual variant calls to diplotypes to milliseconds while reducing human error.

For gene haplotype/nomenclature sets additional information can be associated with the label to expand on how the variations in the sequence will impact the protein functions. Examples of such would be the *1 sequence for CYP2D6 characterized as the reference sequences, while the *4 sequence contains a variation that prevents the protein function by breaking the genomic-protein translation encoding. The CYP2D6*10 sequence contains a variation that only decreases its function, if the *1 reference has an arbitrary activity of 1, then the *10 would have an activity of 0.2.

In order to be of broad clinical use, scalable, automated systems are needed for imputation of function and/or activity of ADMER genes, with return of results to support clinical guidance for drug, dose and exposure for individual patients. At present about 100 ADMER genes are relevant for such guidance and can be found at http://pharmaadme.org/. Of these, CYP2D6 is the most technically difficult to diplotype. Described herein is a system for scalable, automated derivation of diploid functional alleles from unphased Whole Genome Sequencing (WGS) with validation of analytic specificity for CYP2D6.

CYP2D6 is an enzyme of drug bioactivation and elimination. Specifically, CYP2D6 contributes to hepatic metabolism of −25% of drugs in clinical use, including many antidepressants, antipsychotics, opioids, antiemetics, anti-arrhythmics, (3-blockers, cancer chemotherapeutics, and drugs of abuse. The enzymatic activity of CYP2D6 varies widely among individuals, based on level of expression and on functional genomic variations (alleles), resulting in significant clinical consequences for drug metabolism and individual risk of adverse events or drug efficacy.

There is a strong need for timely CYP2D6 activity information to guide the choice of pharmaceutical within and between classes of drugs where therapeutic alternatives exist, and for selection of initial dose. The latter is especially important in pediatric practice, where FDA-labeled dosing guidance is often absent, efficacy is unproven and toxicity is concerning. This is exacerbated in acutely ill newborns, where expression patterns of cytochrome P450 enzymes are still maturing, and polypharmacy is nonnative, with potential for adverse drug-drug interactions with respect to those expression patterns. Fifty-two of the subjects tested herein were acutely ill infants in a neonatal intensive care unit (NICU) who received rapid whole genome sequencing for differential diagnosis of a likely single gene disease. Genetic diseases and congenital anomalies are the leading cause of death in infants, in NICUs, and pediatric intensive care units (PICU). Rates of diagnosis of genomic diseases within this population by rapid whole genome sequencing are as diagnosis, when combined with concomitant return of actionable pharmacogenomics secondary findings, appears to offer the molecular information needed for cogent implementation of precision perinatology. As discussed below activity scores can be provided as potentially actionable, secondary findings in diagnostic WGS reports for a modest increment in cost. While not included in the current American College of Medical Genetics guidelines, a panel of pharmacogenomic activity scores fits well with the more recent American Society of Human Genetics guidelines with respect to reporting of secondary findings in infants and children.

Pharmaceutical choice and initial dose selection is crucial in children with neurodevelopmental disabilities for whom CYP2D6 substrates, such as aripiprazole, atomoxetine, citalopram, fluoxetine, fluvoxamine, and risperidone, are commonly prescribed. Children with developmental disabilities are uniquely vulnerable to the limitations of subjectively guided medication management, the mainstay of current practice, screening for side-effects, and assessment of target symptoms such as anxiety and irritability. Exome and genome sequencing of children with neurodevelopmental disabilities for etiologic diagnosis is starting to become the standard of care in light of recent reports showing rates of diagnosis of single gene disorders of 31-47% in this population. For this group, automated return of actionable pharmacogenomic secondary findings in diagnostic WGS reports is highly desirable for implementation of precision medicine.

Specific pharmaceutical selection within a class is especially important when the therapeutic index is narrow, and in indications where biological responses take weeks or months to measure. This is exemplified by the selective serotonin reuptake inhibitors for young children, with poorly defined starting dose, compounded by parent comfort level, and provider experience. Dose adjustments are based largely on parent and teacher impressions of medication tolerance and effect, requiring 4 weeks post initiation of treatment. Self-reports in pediatric populations may be absent or difficult to interpret. Individuals with alleles that increase CYP2D6 activity at standard starting dose result in lower than expected drug levels and risk treatment failure, not apparent clinically until at least one month into treatment. Conversely poor metabolizers may have toxicity at typical doses, resulting in risk of serotonin syndrome, or increased risk of known adverse reactions including suicidal ideation, activation, and treatment induced mania. For these reasons genotype-aided dosing is increasingly being recognized as important.

Despite the central importance for clinical pharmacogenomics and precision medicine, there is not a current uniform standard for clinical determination of CYP2D6 diplotypes, nor ready translation into clinically actionable results. The most accurate method to produce CYP2D6 diplotypes result from expensive and tedious manual integration of results from copy number assays, a panel of genotypes, and Sanger sequences of long-range genomic PCR products. Genotypes require onerous translation from genomic coordinates into the pharmacogenomic star allele format, and, expert inference of the associated functional activity preventing utilization in the clinical setting. These steps require considerable knowledge of details regarding genome sequence nomenclature and conventions, CYP2D6 haplotype (star allele) nomenclature, and CYP2D6 haplotype-CYP2D6 phenotype relationships. Furthermore, mappings between these are not necessarily intuitive, one-to-one or fixed with respect to time, greatly limiting the practicality of general adoption of interpretation of CYP2D6 genomic results without computational methods. Finally, the current methods are too slow to guide acute clinical decision making. Although computational methods are being developed to assess CYP2D6 genotype from high throughput sequence data, the system of the present invention is advantageous as a homogenous method that is rapid, scalable and has minimal incremental cost in the setting of a whole genome sequence. Furthermore, the system of the present invention has minimal requirement for expert domain knowledge for operation, since it performs the intermediate mapping, translation and inference steps.

Given the complexity of variation in CYP2D6, the variable quality of haplotype definitions, and broad types of variation seen in the samples, the system of the present invention performed well. The clinical sensitivity of one embodiment of the system of the present invention was 93.4% (an activity score was assigned for 57 of 61 subjects), compared with 98.4% (60 of 61) with the integrated results of three consensus reference methods. Critically, the clinical specificity of the system of the present invention was 98.2% (56 of 57 Activity Scores were concordant with the consensus reference). Although the samples tested and described later herein represented the diversity and complexity of CYP2D6 nucleotide and structural variation, they did not include all possible haplotypes.

For CYP2D6, the most polymorphic ADMER locus, the current complete diplotype set contained 8,128 entries. The remaining ˜99 ADMER genes are considerably less complex. While clinical validation for ˜100 genes is onerous, in silico mapping may reduce that burden to a small subset of structural variations and gene—pseudogene instances where empiric evidence is needed.

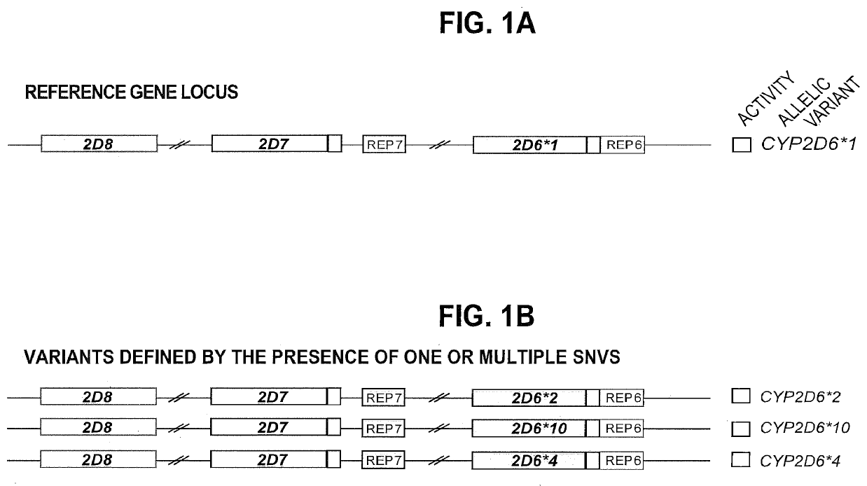

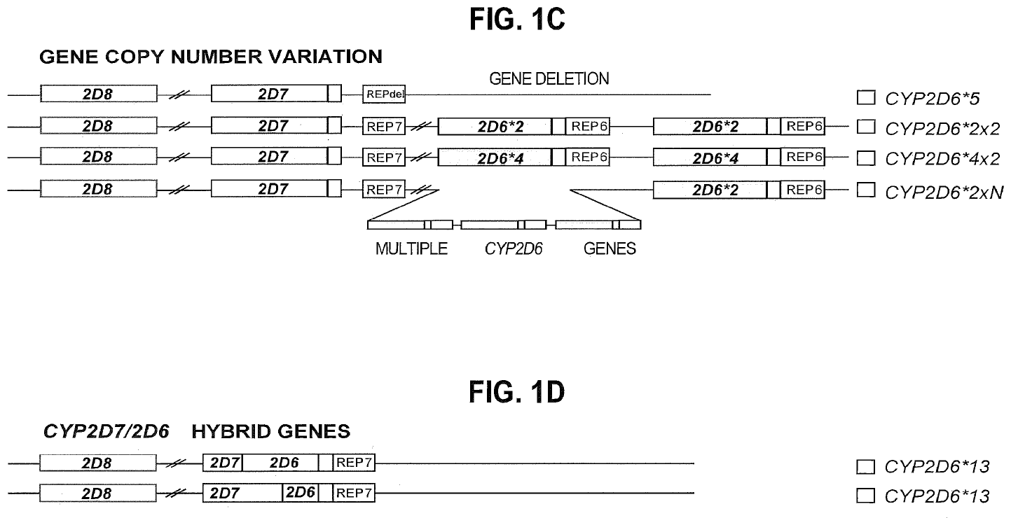

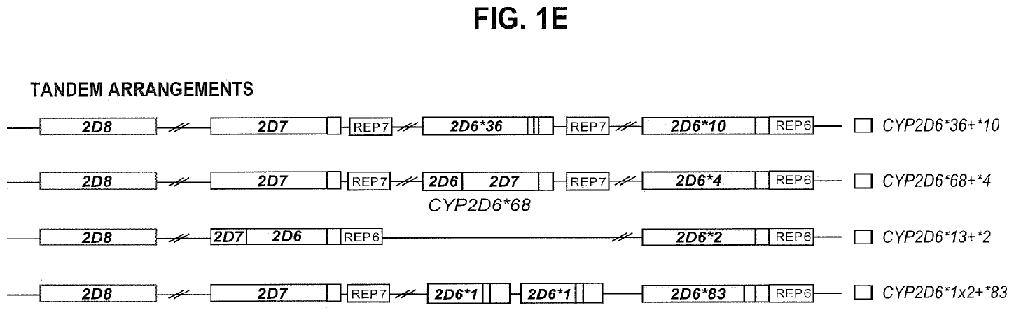

The region of human chromosome 22 to which CYP2D6 maps is highly polymorphic. In addition to CYP2D6, the 37 kb region contains a homologous, nonfunctional gene that arose through gene duplication (CYP2D7), and a pseudogene that arose through gene conversion (CYP2D8). The CYP2D region also contains two, Alu-rich, 2.8 kb repeated regions (REP6 and REP7) which are substrates for a wide variety of common structural variations of CYP2D6, including copy number variations or CNVs, gene conversions, rearrangements, and combinations thereof shown in FIG. 1. CYP2D6 also features hundreds of nucleotide variants, many of which alter enzymatic activity. Given this complexity, the routine clinical determination of individual CYP2D6 activity by genomic analysis remains challenging. Costly and labor intensive, integration and interpretation of nucleotide genotypes, structural variant analysis, copy number determinations, and, in some cases, Sanger sequencing, are necessary to unambiguously identify the specific combination of two haplotypes (diplotype) that is predictive of an individual's CYP2D6 activity.

FIG. 1 provides a depiction of the structure of the highly polymorphic CYP2D6/2D7/2D8 locus, showing the relative activity of CYP2D6 for the reference and 13 variant haplotypes. Specifically, Panel A depicts the reference Chr 22 locus comprising the CYP2D6*1 haplotype (white) and two non-functional, parologs, CYP2D7 (red) and CYP2D8 (gray). Note that the locus is on the minus strand and is shown in reverse. REP6 and REP7 are paralogous, Alu-containing, 600 bp repetitive segments found downstream of CYP2D6 and CYP2D7, respectively. The blue boxes indicate identical unique sequences downstream of CYP2D6 and CYP2D7, separated from REP7 by 1.6 kb in the latter. Panel B shows three CYP2D6 haplotypes (CYP2D6*2,CYP2D6*10, and CYP2D6*4), which are defined by the presence of specific sets of nucleotide variations. The CYP2D6 activity conveyed by these haplotypes are shown by boxes, where green is normal, orange has decreased activity, red is non-functional, and blue has increased activity. Panel C shows the most common CYP2D6 copy number variations. CYP2D6*5 is characterized by deletion of CYP2D6 and fusion of REP6 and REP7 (REP-DEL). Duplication haplotypes have two or more CYP2D6 copies, as exemplified by CYP2D6*2×2 (ultrarapid metabolizer) and CYP2D6*4×2 (non-functional). Less common are copy number variants with 3 or more copies. Duplications have also been reported for CYP2D6 sequences containing nucleotide variations. Panel D shows hybrid genes composed of CYP2D7 and CYP2D6 fusion products that result from unequal recombination. A number of hybrid genes with a variety of switch regions have been described and are consolidated as the CYP2D6*13 haplotype. Panel E shows four tandem arrangements, featuring two or more, non-identical copies of CYP2D6.

Case Study Example and Results

Genomic samples from 61 subjects were chosen for analysis. They included seven HapMap subjects (NA12878, NA12877, NA12882, NA07019 and NA12753, NA18507 and NA19685 of which NA12878, NA12877 and NA12882) were a familial trio. Retrospective samples, UDT002 and UDT173, were from a validation set with known molecular diagnoses for genomic diseases. 26 acutely ill infants were enrolled in the study, of which 13 were singleton probands and 13 were familial trios (proband infant and both parents). Probands were suspected of having a monogenomic disease, but without a definitive diagnosis at time of enrollment. Subject ethnicity and relatedness are shown in Table 1 below.

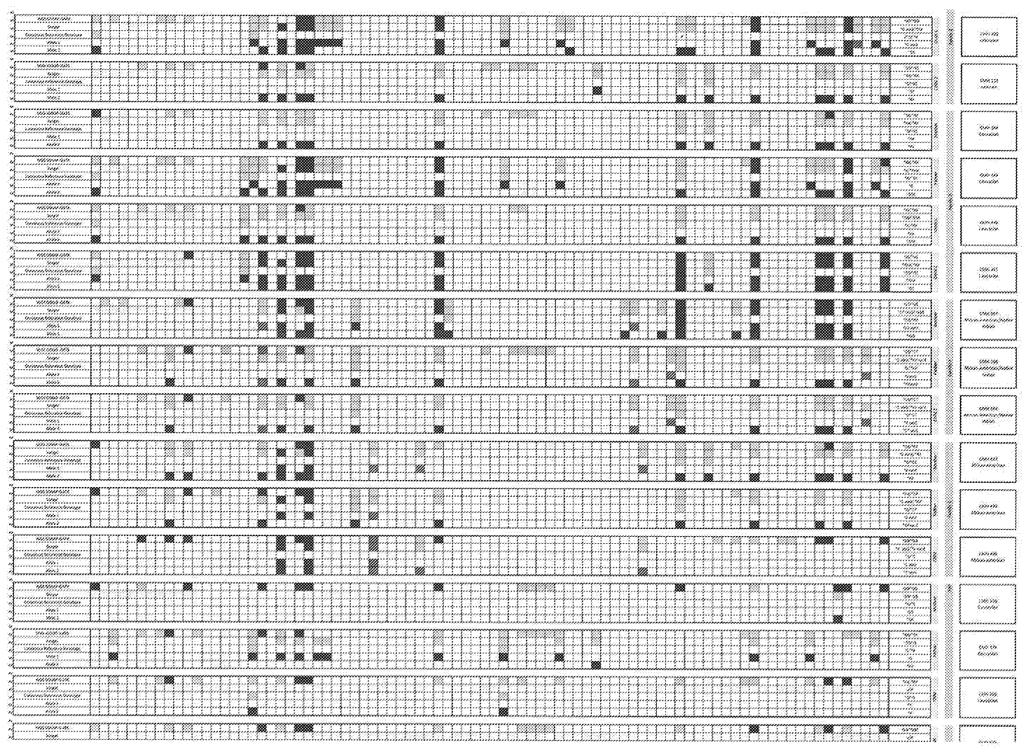

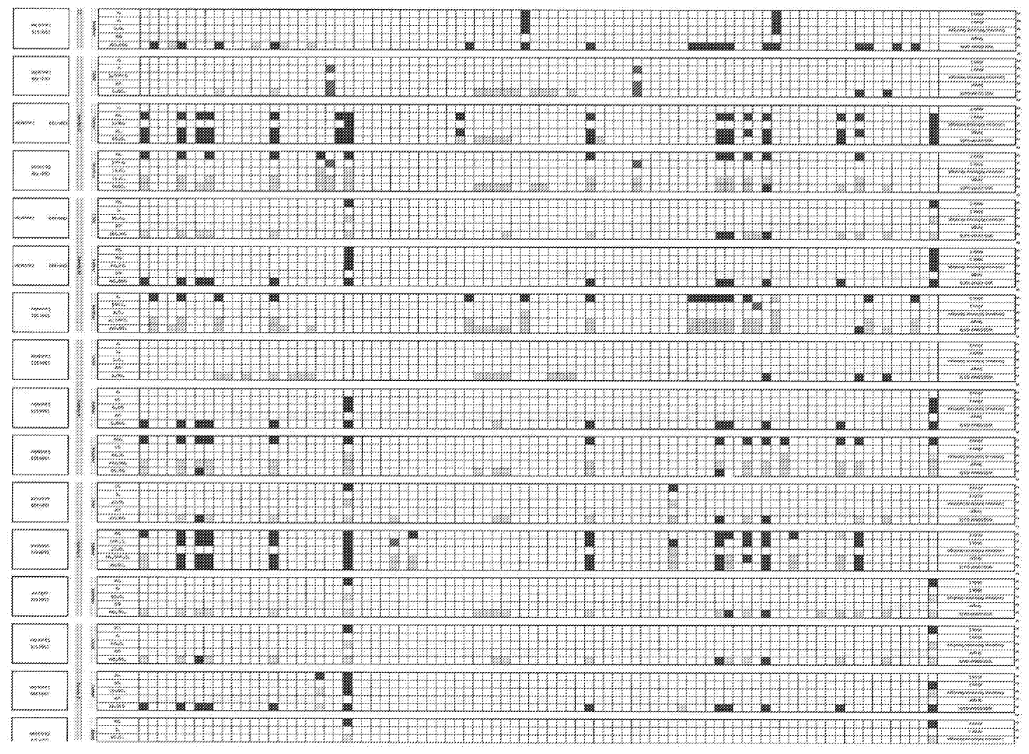

Table 1 below summarizes diplotypes and activity score assignments and phenotype prediction for different reference methods. TaqMan® refers to genotype analysis using a panel of genotyping assays interrogating selected more common SNPs (see FIG. 5). Copy Number Variation or CNV refers to quantitative multiplex PCR performed on the CYP2D6 to determine gene copy number (deletion, duplication, multiplication and gene hybrids). This assay was complemented by XL-PCR amplifying the entire duplicated or hybrid gene copies and subsequent genotyping by TaqMan® and/or sequencing to determine which allele carries the CNV. The table shows the number of gene copies detected and whether CYP2D6/CYP2D7 gene hybrids (6/7 hyb) structures were identified. Sanger refers to diplotype calls based on Sanger sequencing of a 6.6 kb long XL-PCR product encompassing the CYP2D6 gene. Consensus reference indicates calls derived from a combination of CNV, TaqMan® and Sanger sequencing. The system of the present invention (denoted as Constellation in Table 1 below) refers to calls made by the system of the present invention using vcf files generated from WGS. Activity Scores (AS) were assigned to diplotypes derived from the consensus reference diplotypes and the system of the present invention. Inconsistent calls between the consensus reference calls and the system of the present invention are bolded. Phenotype prediction is consistent between the consensus reference call and the system of the present invention (denoted as Constellation in Table 1 below) with the exception of three cases. UM, EM, IM and PM indicate ultrarapid, extensive, intermediate and poor metabolizer phenotypes, respectively. (+) denotes that the subject was identified as having a duplication. [mac], multiple ambiguous calls causing a ‘no call’ result. #novelsubvariant(s) identified (see FIG. 5 for details). For brevity, this is only annotated in the column labeled ‘Sanger’. [*2], TaqMan® genotype result for SNP rs16947 was not conclusive. Allele subtype assignments are not shown in this table, but provided for each individual in FIG. 5. Subjects with a CMH-prefix are patient samples, those with a NA-prefix were obtained from the Coriell Institute. Relatedness of subjects is as indicated. No, not related; M, mother; F, father; C, child; C-1 and C-2; child 1 and child 2.

TABLE 1

| | | | CYP206 | | | Consen- | | Consen- | | Pheno- |
|---|---|---|---|---|---|---|---|---|---|---|
| | Relat- | Ethnic- | gene copy | TaqMan | Sanger | sus | Constella- | sus | Constella- | type |
| Subject ID | ed | ity | number | Sequencing | Sequencing | Reference | tion | Reference | tion | prediction |
| CMH 064 | no | C | 1 | *35/*35 | *35/*35 | *5/*35 | *5/*35 | 1 | 1 | EM |
| CMH 076 | no | AA | 2 | *2/*2 | *2/*2var1 | *2/*2 | *2/*2 | 2 | 2 | EM |
| CMH 172 | no | Mex | 2 | *1/*1 | *1/*1 | *1/*1 | *1/*1 | 2 | 2 | EM |
| UDT 002 | no | n/a | 2 + 617 | *4/*4 | *4/*4 # | *4/*68 + *4 | *4/*4 | 0 | 0 | PM |
| | | | hyb | | | | | | | |
| UDT 173 | no | n/a | 2 + 617 | *1/*4 | *1/*4 # | *1/*68 + *4 | *1/*4 | 1 | 1 | EM |
| | | | hyb | | | | | | | |
| CMH 557 | no | C | 2 | *1/*1 | NO | *1/*1 | *1/*1 | 2 | 2 | EM |
| CMH 563 | no | C | 2 | *1/*2 | NO | *1/*2 | *1/*2 | 2 | 2 | EM |
| CMH 010 | no | C | 2 | *1/*41 | NO | *1/*41 | *1/*41 | 1.5 | 1.5 | EM |
| CMH 154 | no | C | 2 | *1/*41 | NO | *1/*41 | *1/*41 | 1.5 | 1.5 | EM |
| CMH 487 | no | C | 2 | *1/*35 | NO | *1/*35′ | *1/*35 | 2 | 2 | EM |
| CMH 545 | no | C | 2 | *1/*4 | NO | *1/*4 | *1/*4 | 1 | 1 | EM |
| CMH 589 | no | C | 2 | *4/*4 | *4/*4 * | *4/*4 | *4/*4 | 0 | 0 | PM |
| CMH 663 | no | C | 2 | *4/*41 | NO | *4/*41 | *4/*41 | 0.5 | 0.5 | IM |
| CMH 677 | no | C | 2 | *4/*4 | NO | *4/*4 | *4/*4 | 0 | 0 | PM |
| CMH 731 | no | C | 2 | *4/*10 | *4/*10 | *4/*10 | [0] | 0.5 | no call | IM |
| NA07019 | no | C | 2 | *1/*4 | NO | *1/*4 | *1/*4 | 1 | 1 | EM |
| NA12753 | no | C | 2 | *2/*3 | NO | *2/*3 | *2/*3 | 1 | 1 | EM |
| NA19685 | no | Mex | 3 | *1/*2 | NO | *1/*2 × 2 | *1/*2(*) | 3 | 3 | UM |
| NA18507 | no | Yoruban | 3 | *2/*4 | *2/*4 | *2/*4 × 2 | [0](*) | 1 | no call | EM |
| CMH 186 | M | Mex | 2 + 617 | *2/**4 | *2/*4 # | *2/*68 + *4 | *2/*4 | 1 | 1 | EM |
| | | | hyb | | | | | | | |
| CMH 202 | F | Mex | 2 | *4/*45cr | *4/*45 | *4/*45 | *4/*45 | 1 | 1 | EM |
| | | | | 46 | | | | | | |
| CMH 184 | C-1 | Mex | 2 | *2/*4 | *2/*4 | *2/*4 | *2/*4 | 1 | 1 | EM |
| CMH 185 | C-2 | Mex | 2 + 617 | *4/**4 | *4/*4 # | *4/*68 + *4 | *4/*4 | 0 | 0 | PM |
| | | | hyb | | | | | | | |
| CMH 224 | M | n/a | 2 | *4/*41 | *4/*41 | *4/*41 | *4/*41 | 0.5 | 0.5 | IM |
| CMH 222 | C-1 | n/a | 2 | [*2]/*4 | *4/*59 # | *4/*59 | *4/*59 | 0.5 | 0.5 | IM |
| CMH 223 | C-2 | n/a | 2 | *1/*41 | *3.3/*41 | *1/*41 | *33141 | 1.5 | 1.5 | EM |
| CMH 248 | M | C | 2 | *1/*41 | *1A/*41 | *1/*41 | *1/*41 | 1.5 | 1.5 | EM |
| CMH 249 | F | C | 2 | *4/*35 | *4/*35 | *4/*35 | *4/*35 | 1 | 1 | EM |
| CMH 446 | C-1 | C | 2 | *1/*35 | *1A/*35 | *1/*35 | *1/*35 | 2 | 2 | EM |
| CMH 447 | C-2 | C | 2 | *35² *41 | *35A² *41 | *35² *41 | *35² *41 | 1.5 | 1.5 | EM |
| CMH 397 | M | AA/A1 | 2 | *17/*45 | *17/*45 # | *17/*45 | *17/*45 | 1.5 | 1.5 | EM |
| CMH 398 | F | AA/A1 | 2 | *1/*17 | *1/*17 # | *1/*17 | *1/*17 | 1.5 | 1.5 | EM |
| CMH 396 | C | AA/A1 | 2 | *1/*17 | *1/*17 # | #1/*17 | *1/*17 | 1.5 | 1.5 | EM |
| CMH 437 | M | AA | 2 | *1/*41 | *1/*41 # | *1/*41 | *1141 | 1.5 | 1.5 | EM |
| CMH 438 | F | AA | 2 | *1/*17 | *1var2/*17 # | *1/*17 | *1/*17 | 1.5 | 1.5 | EM |
| CMH 436 | C | AA | 2 | *1/*1 | *1/*1 # | *1/*1 | *1/*1 | 2 | 2 | EM |
| CMH 570 | M | C | 2 | *1/*1 | *1/*1 | *1/*1 | *39/*95 | 2 | unknown | EM |
| CMH 571 | F | C | 2 | *1/*4 | *4/*33 | *1/*4 | *4/*33 | 1 | 1 | EM |
| CMH 569 | C | C | 2 | *1/*4 | NO | *1/*4 | * 1/*4 | 1 | 1 | EM |
| CMH 579 | M | C | 2 | *1/*2 | NO | *1/*2 | *1/*2 | 2 | 2 | EM |
| CMH 580 | F | C | 2 | *2/*41 | NO | *2/*41 | *2/*41 | 1.5 | 1.5 | EM |
| CMH 578 | C | C | 2 | *1/*2 | NO | *1/*2 | *1/*2 | 2 | 2 | EM |
| CMH 630 | M | n/a | 2 | *1/*2 | NO | *1/*2 | *1/*2 | 2 | 2 | EM |
| CMH 631 | F | n/a | 2 | *2/*17 | *17/*84 # | *2/*17 | *17/*84 | unknown | unknown | unknown |
| CMH 629 | C | MR | 2 | *1/*17 | NO | *1/*17 | *1/*17 | 1.5 | 1.5 | EM |
| CMH 673 | M | C | 2 | *1/*35 | *1/*35 | *1/*35 | *35² *83 | 2 | 1 | EM |
| CMH 674 | F | C | 1 | *2/*2 | NO | *2/*5 | *2/*5 | 1 | 1 | EM |
| CMH 672 | C | C | 1 | *1/*1 | NO | *1/*5 | *1/*5 | 1 | 1 | EM |
| CMH 681 | M | C | 2 | *1/*4 | *1/*4 # | *1/*4 | *1/*4 | 1 | 1 | EM |
| CMH 682 | F | C | 2 | *2/*2 | NO | *2/*2 | *2/*2 | 2 | 2 | EM |
| CMH 680 | C | C | 2 | *1/*2 | NO | *1/*2 | *1/*2 | 2 | 2 | EM |
| CMH 729 | M | C | 2 | *1/*41 | *1/*41 # | *1/*41 | *1/*41 | 1.5 | 1.5 | EM |
| CMH 730 | F | C | 1 | #2/*2 | *59/*59 | *5/*59 | *5/*59 | 0.5 | 0.5 | IM |
| CMH 728 | C | C | 1 | *1/*1 | *1var5/*5 # | *1/*5 | *1/*5 | 1 | 1 | EM |
| CMH 679 | M | C | 2 | *4/*4 | NO | *4/*4 | *4/*4 | 0 | 0 | PM |
| CMH 678 | C | C | 2 | *1/*4 | NO | *1/*4 | *1/*4 | 1 | 1 | EM |
| CMH 719 | M | C | 2 | *1/*2 | NO | *1/*2 | *1/*2 | 2 | 2 | EM |
| CMH 718 | C | C | 2 | *1/*2 | NO | *1/*2 | *1/*2 | 2 | 2 | EM |
| NA12878 | M | Eur | 2 + 617 | *3/*4 | *3/*4 | *3/*68 + *4 | #3/*4 | 0 | 0 | PM |
| | | | hyb | | | | | | | |
| NA12877 | F | Eur | 2 + 617 | *3/*4 | *4/*4 | *4/*68 + *4 | *4/*4 | 0 | 0 | PM |
| | | | hyb | | | | | | | |
| NA12882 | C | Eur | 2 + 617 | *3/*4 | *4/*4 | *4/*68 + *4 | *4/*4 | 0 | 0 | PM |
| | | | hyb | | | | | | | |

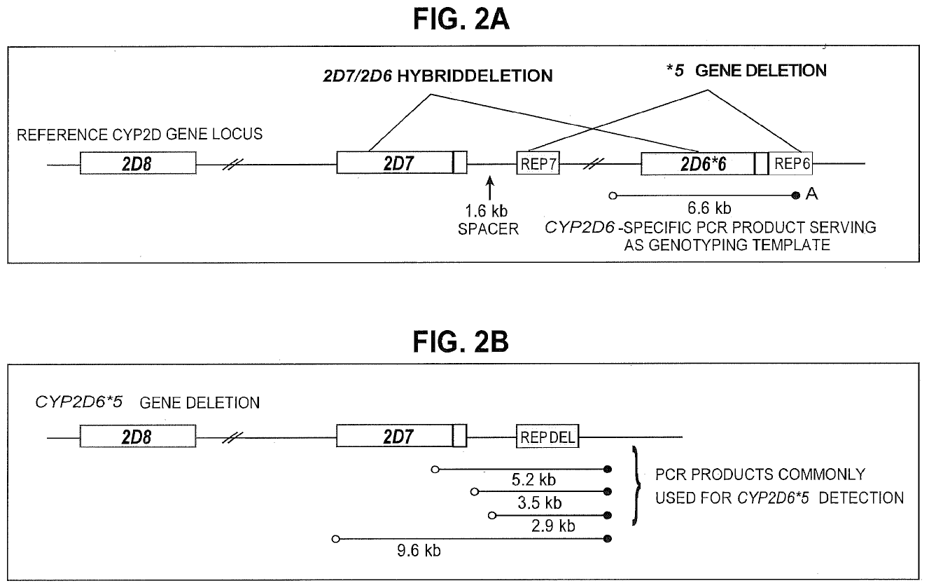

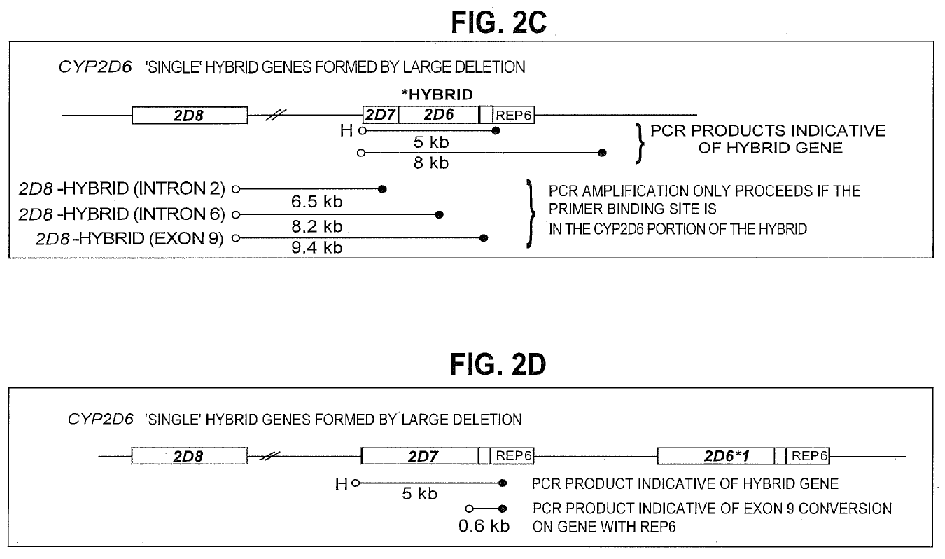

CYP2D6 genotyping was performed in accordance with known practices. Generally, long-range PCR was used to amplify a 6.6 kb fragment encompassing the CYP2D6 (fragment A), a 3.5 kb fragment from the intergenic region of CYP2D6 duplication structures (fragment B), and a 5 kb fragment from CYP2D7/2D6 hybrid structures (fragment H). Presence of fragments was determined by band visualization following agarose gel electrophoresis. The gene regions amplified are shown in FIG. 2.

FIG. 2 depicts long-range PCR products used to define CYP2D7/2D6 hybrid genes. CYP2D6, CYP2D7, and CYP2D8 genes are shown in white, red, and dark gray boxes, respectively. The 600-bp repeat element immediately downstream of CYP2D6 and CYP2D exon 9 is shown in blue. Alu repetitive elements (REP) are in red and light gray; REP-DEL indicates a fused repeat element generated by a large deletion involving parts of those elements from both genes. PCR fragments denote primer specifically to CYP2D6 and CYP2D7. (A) Graph represents the CYP2D reference locus. Areas affected by large deletions and implicated in CYP2D7/2D6 hybrid formation and the CYP2D6*5 gene deletion are as indicated. (B) Graphic display of the CYP2D6*5 gene deletion allele. Long-range PCR amplicons utilized for detection are shown. (C) Graphic display of CYP2D7/2D6 hybrid genes and their detection by amplification of fragment H. Other depicted fragments are only amplified, if respective rearrangements are present in a sample. (D) Representation of an allele with a CYP2D7 gene lacking the 1.6-kb spacer. This CYP2D7 variant also supports formation of fragment H although the CYPD7/2D6 switch occurs in the downstream region.

To test for single nucleotide variations, amplicons were diluted 2000-fold and used in TaqMan® genotyping assays (Applied Biosystems, Foster City, Calif.) to detect the following CYP2D6 (NM_000106.5) sequence variations: c.31G>A (rs769258), 100C>T (rs1065852), 124G>A (rs5030862), 883G>C (rs5030863), 1023C>T (rs28371706),1707delT (rs5030655), 1716G>A (rs28371710), 1846G>A (rs3892097), 2549delA (rs35742686), 2615delAAG (rs5030656), 2850C>T (rs16947), 2935A>C (r55030867), 2988G>A (rs28371725), 3183G>A (r559421388), 3259insGT (rs72549346), and 4042G>A (rs112431047) allowing us to assign haplotypes defined as CYP2D6*2, *3, *4, *6, *7, *9, *10, *11, *17, *29, *31, *35, *41, *42, and*45. In the absence of these variants, the haplotype assigned was CYP2D6*1. If the haplotype could not be determined unequivocally, the most parsimonious approximation was assigned. CYP2D6 duplications/multiplications, the CYP2D6*5 gene deletion, CYP2D7/2D6 hybrid arrangements (collated under the CYP2D6*13 designation, and other CYP2D6/2D7 hybrids (such as CYP2D6*68), were identified by quantitative CNV assay and confirmed by long-range PCR. Furthermore, duplicated gene copies were genotyped by performing TaqMan® genotyping assays on an XL-PCR product, (fragment D) that encompasses the entire duplicated gene copy.

An Activity Score was assigned to each allele with the traditional phenotype classifications (poor (PM), intermediate (IM), extensive (EM) and ultrarapid (UM) metabolizers) in accordance with guidelines from the Clinical Pharmacogenetics Implementation Consortium.

The following analysis uses Sanger sequencing. The CYP2D6 locus, including at least 600 and 150 nucleotides upstream and downstream of the translation start and stop codons, respectively, was sequenced in both directions. As shown in FIG. 2 the 6.6 kb CYP2D6 fragment was purified with a PCR clean-up kit. Sequencing was performed on a 3730x genomic analyzer. Sequences were assembled using Sequencer software V4.9 and compared to the CYP2D6 accessions M33388.1 and AY545216.

To determine the haplotypes of two novel subvariants of known CYP2D6 haplotypes in subject CMH396, allele-specific XL-PCR was perfoiined with primer −740C>T to generate a 5.5 kb XL-PCR product from the CYP2D6*1. WGS was performed using known methods. Generally, 500 ng of DNA was sheared, end repaired, A-tailed and adaptor ligated. PCR was omitted. The libraries were purified using SPRI beads and quantitation was performed using real-time PCR. The libraries were denatured using 0.1M NaOH and diluted to 2.8 pM in hybridization buffer.

Samples for WGS were sequenced on HiSeq 2500 instruments (Illumina) on rapid run or high throughput mode to a depth of −120 GB with 2×100 nt reads. Samples were aligned and variants called with Genomic Short-read Nucleotide Alignment Program (GSNAP) and the Genome Analysis Tool Kit (GATK) relative to the GRCh37 CYP2D6*2 reference, yielding 5.1 million variants per genome as a .vcf file (Table 2). Subsequently, variants were compared to the standard CYP2D6*1 reference (AY545216) allele.

TABLE 2

| | | | Aligned | Aligned | | | |
|---|---|---|---|---|---|---|---|
| | | | sequences that | sequences | | ACMG-like | |
| | | Total | passed filters | with Q | | category 1-3 | Rare category |
| Sample | Total reads | sequence (GB) | (GB) | score >20 | Total variants | variants | 1-3 variants |
| CMH000064 | 1,209,959,172 | 122 | 116 | 108 | 5,038,698 | 3379 | 557 |
| cmh000076 | 1,342,226,410 | 135 | 130 | 121 | 5,619,234 | 4172 | 1534 |
| cmh000172 | 1,133,464,063 | 114 | 111 | 105 | 4,953,813 | 3268 | 861 |
| UDT_002 | 1,474,588,253 | 147 | 138 | 122 | 4,882,585 | 3352 | 634 |
| UDT_173 | 1,600,532,150 | 160 | 148 | 128 | 5,112,752 | 3550 | 674 |
| CMH000557 | 2,431,401,972 | 245 | 231 | 213 | 5,126,705 | 3714 | 612 |
| CMH000563 | 1,086,099,138 | 109 | 101 | 94 | 4,950,650 | 3525 | 593 |
| cmh000010 | 1,014,606,386 | 127 | 121 | 114 | 4,893,386 | 3126 | 612 |
| cmh000154 | 1,362,225,100 | 137 | 129 | 114 | 4,483,137 | 2243 | 545 |
| cmh000487 | 984,302,114 | 99 | 90 | 81 | 4,777,125 | 2963 | 634 |
| CMH000545 | 1,299,071,626 | 131 | 123 | 112 | 5,012,489 | 3405 | 669 |
| cmh000589 | 1,063,276,174 | 107 | 101 | 95 | 4,965,301 | 4093 | 648 |
| cmh000663 | 1,159,263,976 | 117 | 109 | 97 | 4,962,407 | 3274 | 608 |
| cmh000677 | 1,109,230,876 | 112 | 106 | 98 | 5,023,671 | 3372 | 597 |
| cmh000731 | 1,539,656,776 | 155 | 149 | 139 | 5,186,787 | 3764 | 689 |
| NA07019 | 1,013,773,530 | 127 | 122 | 115 | 4,907,336 | 3031 | 606 |
| NA12753 | 1,159,856,750 | 146 | 137 | 126 | 5,033,116 | 3859 | 772 |
| cmh000186 | 1,204,702,734 | 120 | 115 | 104 | 4,965,565 | 3311 | 792 |
| cmh000202 | 1,241,622,263 | 124 | 118 | 106 | 4,983,097 | 3539 | 890 |
| cmh000184 | 1,539,534,606 | 153 | 143 | 124 | 4,956,398 | 3568 | 910 |
| cmh000185 | 1,252,265,788 | 125 | 119 | 107 | 4,961,672 | 3355 | 833 |
| cmh000224 | 1,234,986,528 | 124 | 121 | 113 | 5,013,492 | 3382 | 608 |
| cmh000222 | 1,122,304,294 | 113 | 110 | 104 | 5,027,846 | 3477 | 599 |
| cmh000223 | 1,112,689,845 | 112 | 109 | 102 | 4,998,397 | 3297 | 566 |
| cmh000248 | 1,152,234,751 | 116 | 111 | 104 | 5,105,450 | 3342 | 661 |
| cmh000249 | 1,115,963,861 | 112 | 109 | 103 | 5,027,304 | 3192 | 559 |
| cmh000446 | 1,114,747,660 | 112 | 109 | 102 | 5,073,908 | 3312 | 611 |
| cmh000447 | 1,280,811,247 | 129 | 125 | 116 | 5,230,528 | 3502 | 772 |
| cmh000397 | 1,141,378,626 | 115 | 112 | 106 | 6,015,080 | 5063 | 2407 |
| cmh000398 | 1,064,489,375 | 107 | 104 | 98 | 5,820,501 | 4732 | 2165 |
| cmh000396 | 1,125,193,331 | 113 | 110 | 104 | 5,875,359 | 4921 | 2266 |
| cmh000437 | 1,232,107,098 | 124 | 117 | 107 | 5,904,474 | 4984 | 2361 |
| cmh000438 | 1,182,378,536 | 119 | 110 | 100 | 5,590,365 | 4545 | 2438 |
| cmh000436 | 1,239,018,816 | 125 | 115 | 99 | 5,763,073 | 4913 | 2387 |
| cmh000570 | 557,567,858 | 56 | 53 | 47 | 4,198,715 | 1981 | 481 |
| cmh000571 | 868,335,656 | 87 | 64 | 53 | 4,416,758 | 2242 | 481 |
| CMH0000569 | 995,793,286 | 100 | 81 | 67 | 5,040,253 | 3325 | 739 |
| cmh000579 | 574,273,929 | 58 | 56 | 50 | 4,249,153 | 1950 | 493 |
| cmh000580 | 1,187,117,200 | 119 | 114 | 107 | 4,990,860 | 3489 | 652 |
| cmh000578 | 1,016,894,441 | 102 | 96 | 85 | 4,763,591 | 2859 | 538 |
| cmh000630 | 1,191,000,920 | 120 | 115 | 108 | 5,045,223 | 3486 | 665 |
| cmh000631 | 1,142,908,792 | 115 | 108 | 99 | 5,836,643 | 5179 | 2508 |
| cmh000629 | 1,260,077,897 | 127 | 122 | 113 | 5,548,134 | 4077 | 1573 |
| cmh000673 | 1,180,425,018 | 119 | 107 | 94 | 4,962,776 | 3212 | 628 |
| cmh000674 | 1,046,746,788 | 105 | 101 | 92 | 5,031,716 | 3493 | 695 |
| cmh000672 | 1,338,643,358 | 135 | 127 | 119 | 5,089,539 | 3506 | 648 |
| cmh000681 | 1,244,077,138 | 125 | 121 | 113 | 4,845,930 | 3125 | 605 |
| cmh000682 | 1,287,535,036 | 130 | 125 | 117 | 5,101,798 | 3642 | 668 |
| cmh000680 | 1,236,090,235 | 124 | 116 | 104 | 4,896,283 | 3052 | 583 |
| cmh000729 | 719,347,178 | 72 | 70 | 66 | 4,947,962 | 3242 | 598 |
| cmh000730 | 1,262,547,732 | 127 | 123 | 115 | 5,047,790 | 3607 | 655 |
| cmh000728 | 1,385,506,538 | 139 | 135 | 126 | 5,143,754 | 3774 | 655 |
| cmh000679 | 1,098,098,560 | 110 | 107 | 101 | 5,076,651 | 3483 | 722 |
| cmh000678 | 1,141,745,228 | 115 | 111 | 105 | 5,080,200 | 3439 | 681 |
| cmh000719 | 1,035,135,530 | 130 | 125 | 118 | 4,853,549 | 3664 | 780 |
| cmh000718 | 893,119,414 | 90 | 86 | 76 | 4,752,853 | 2735 | 542 |
| NA12878 | 1,566,327,054 | 156 | 154 | 153 | 4,764,620 | 3342 | 570 |
| NA12877 | 1,494,455,776 | 149 | 147 | 146 | 4,730,735 | 3252 | 568 |
| NA12882 | 1,473,252,906 | 147 | 145 | 144 | 4,747,762 | 3350 | 566 |
| NA18507 | 826,988,034 | 104 | 89 | 82 | 5,403,475 | 5094 | 2860 |
| NA19685 | 905,705,816 | 114 | 96 | 89 | 4,661,021 | 3283 | 914 |
| Average | 1,184,748,871 | 121 | 115 | 1306 | 5,056,876 | 3,531 | 914 |

Data inputs for the system of the present invention were variant call format (.vcf) file, a gene directory with chromosomal position, and nomenclature file for each locus to be diplotyped. The position file contained the location of the gene transcript [Chr:start-stop] according to the GRCh37 reference. The nomenclature file contained the full set of possible genotypes, one per line, in the format [allele_name<tab>var1,var2,var3], with variants annotated as [Chr˜start˜stop˜var]. The output is the most likely diplotype for that sample. The system of the present invention was implemented in the java programming language but other programming languages can be used.

To determine if possible copy number variation was present a BAM file (.bam) and a BED file (.bed) are used. The BAM file contains aligned reads against a reference genome and the BED file containing a list of sentinel regions marked by position against the aligned reference. Sentinel regions are evaluated for depth of coverage as are paired control regions. Significant deviation from expected ratios of coverage indicates the possible presence of copy number variation.

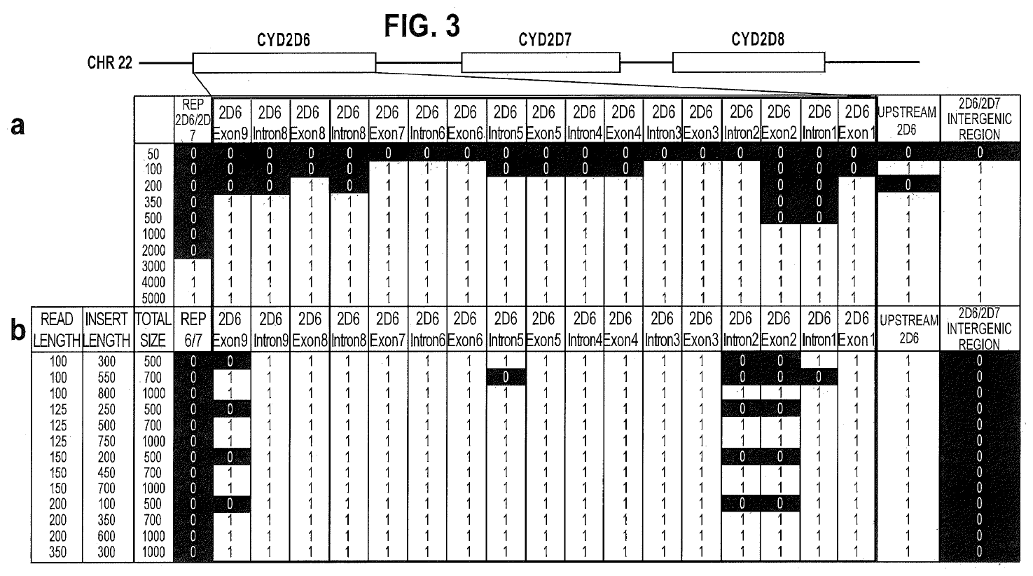

In silico modeling was used to assess whether short read sequences aligned correctly within the CYP2D6 locus. Variant-free reads were tiled across the 37 kb CYP2D6*2 region at 5 nucleotide (nt) spacing and aligned to the CYP2D6*2-containing reference genome (hg19) with the algorithm GSNAP (FIG. 3).

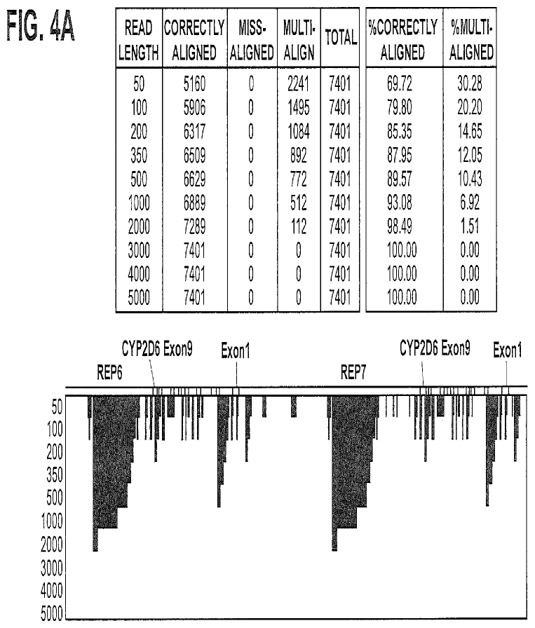

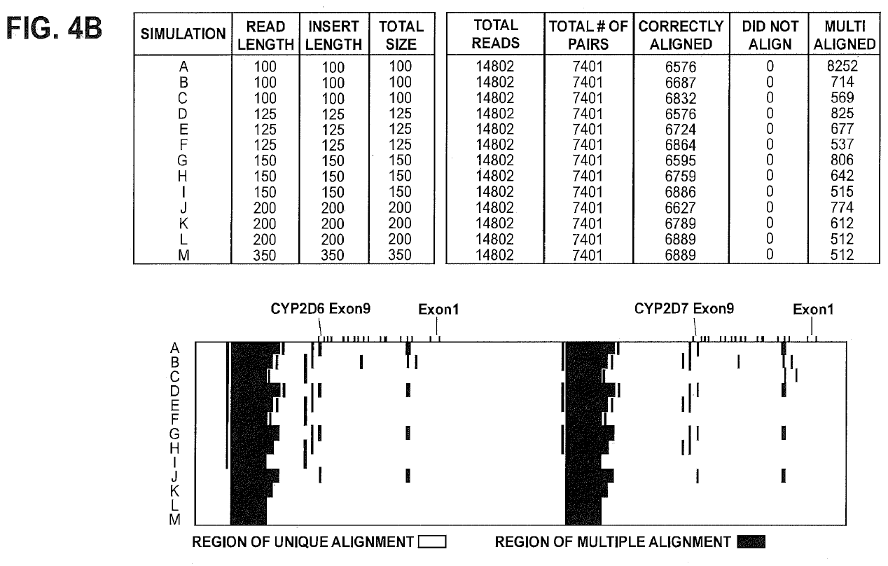

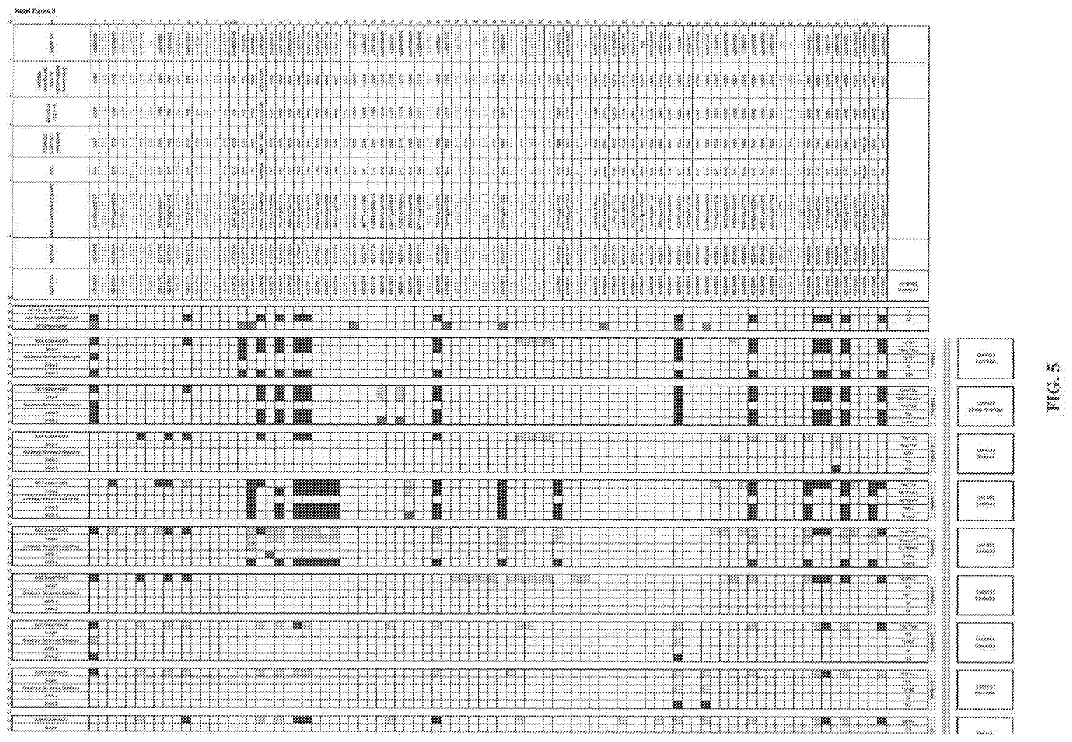

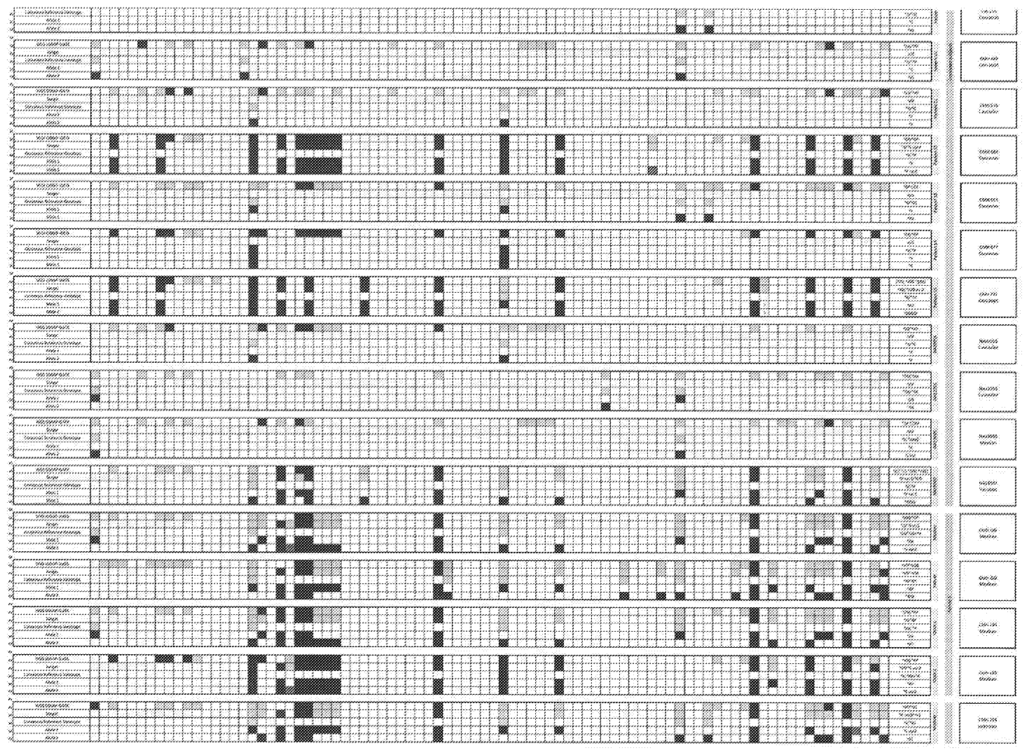

No reads of any size or format misaligned, however, 20% of 100 nt singleton reads aligned ambiguously) (FIG. 4). This was expected based on the high sequence similarity between CYP2D6 and CYP2D7. This ambiguity included CYP2D6 exons required for the determination of functional haplotypes. CYP2D6 exonic ambiguity in alignment resolved at a singleton read length of 500 nt. Exonic and intronic alignment was unique at 1000 nt, and across the entire locus at a read length of 3 kb. Using simulated standard sequencing parameters (paired 100 nt reads separated by 300 nt), CYP2D6 exonic ambiguity was limited to exon 2 (as shown in FIG. 4b). Exonic and intronic alignment ambiguity resolved with 2×100 nt reads separated by 800 nt, 2×125 nt reads separated by 500 nt, or 2×200 nt reads separated by 350 nt. None of these, however, resolved the repetitive regions located upstream and downstream of the CYP2D6 or the CYP2D6/CYP2D7 intergenic region. It should be noted that these models represent an ideal clinical situation without sequencing errors or nucleotide variants. Having determined that alignment to CYP2D6 exons was largely unique with current read lengths (2×100 with 250 nt cassette); the system of the present invention provides an algorithm to impute CYP2D6 diplotypes from WGS. The algorithm is a probabilistic scoring system that computes the score as the noise corrected likelihood that the sequence data matches a particular diplotype. The genotype with the maximum likelihood is then assigned to the individual.

Step 1. For each possible diplotype, compute the noise corrected likelihood based on the observed variants.

Step 2. Then sort the diplotypes in descending order by score, and report the diplotype with the highest score as the most probable.

Such an algorithm is necessary because direct conversion of genotypes to functional alleles is not possible since there is no one-to-one correspondence between a genotype at a nucleotide site, key variants, and an allele, and does not account for copy number variation. Global or local sequence alignment algorithms fail because of noise due both to sequencing errors and variants that are not represented in known/defined CYP2D6 alleles. The latter is particularly crucial since some CYP2D6 allele definitions are based on SNPs in exonic regions rather than complete haplotype sequences. Furthermore, there are no rigorous scoring paths for such that it is difficult to recognize the correct answer among the possible solutions. Thus, the problem is akin to de novo peptide sequencing from tandem mass spectrometry in the presence of false positives and false negatives. A probabilistic scoring system was developed to determine the most likely diplotype match to the WGS-derived.vcf file (Vt) of a test sample, t, based on prior computation of all theoretical haplotypes and corresponding functional alleles (as defined by the Human P450 Nomenclature Committee). For 127 CYP2D6 haplotypes, the defining variant set was determined. The complete set of 8,128 possible diplotypes was generated by combining the variant sets for each pair of haplotypes. For WGS of test sample t, the system of the present invention retrieved the position and zygosity of each variant in the .vcf file, Vt. that was compared with each possible diplotype D1-8128. For a diplotype Da and Vt, X variants were common, Y variants were in Vt only, and Z variants were found in the Da only [X=(Vt∩Da), Y=(Vt−Da), and Z=(Da−Vt)]. A variant location which was homozygous in Vt but heterozygous in the Da set resulted in X+1 and Z+1 score adjustments. A Jaccard similarity coefficient could potentially be used to represent the probability of match P1-8128 of Vt for each Da. However, this assumes variant calling is error free.

To adjust for variant call errors, the scores were adjusted by the sensitivity (lens) and specificity (spec) of WGS variant calling. Assuming independence of variant calls, the score for each variant was reported as a likelihood ratio. For instance, a reported variant (type X) that matched a candidate diplotype was scored as P(Predicted|Present)/P(Predicted|Absent)=Sensitivity/(1−Specificity), type Y scored as (Predicted|Absent)/P(Predicted/Present)=(1−Specificity)/Sensitivity, and type Z scored as P(Not Predicted|Present)/P(Not Predicted|Absent)=(1−Sensitivity)/Specificity. Thus, X was adjusted by A=[sens/(1-spec)], Y adjusted by B=[(1−sens)/spec], and Z adjusted by C=[(1−spec)/sens]. The overall score was the product of likelihood ratios of a diplotype sample set match [score=(A^{x})*(B^{y})*(C^{z})]. A Resultant diplotypes were returned in a reverse sorted list with the highest index, max(P), reported to the output file. The CYP2D6 activity corresponding to the highest scoring diplotype was reported.

In order to assess the ability to align short sequence reads uniquely to their correct location within the CYP2D locus (GRCh37, Chr22:42,518,000-42,555,000), simulated single and paired-end reads were generated from the CYP2D6*2 reference sequence of the 37 kb target region and then mapped to the entire reference genome. CYP2D6*2 region reads were simulated with a quality score of 36, tiling interval of 5 nucleotides, and no mismatches from the reference genome, with sequence coverage of 30× over the target region. Single reads were generated in lengths of 50, 100, 200, 350, 500, 1000, 2000, 3000, 4000, and 5000 nucleotides. Paired-end reads were created with read lengths of 100, 125, 150, 200, and 350 nucleotides and with simulated sequencing library sizes of 500, 750, and 1000 nucleotides for each read length. Each read set was aligned against the GRCh37.p5 reference genome using GSNAP allowing for multiple alignments. Reads which aligned uniquely to their exact position of origin were counted as mappable; reads with unique alignments to incorrect position were labelled as unmappable, and reads which aligned to multiple positions were labeled as ambiguous. Results were compiled for each read set to determine the minimum read size required to resolve the Chr22:42,518,000-42,555,000, with a specific focus on CYP2D6.

To evaluate the performance of one embodiment of the system of the present invention, CYP2D6 alleles were ascertained in 61 samples both by manual integration of results of three conventional methods (quantitative copy number assessment, a panel of TaqMan® genotype assays, and Sanger sequencing of long-range genomic PCR products), and probabilistic WGS analysis by the system of the present invention (Table 1, Table 2 and FIG. 5).The analytic sensitivity and specificity of WGS for nucleotide genotypes with the read alignment and variant calling methods employed was 98.78% and 99.99%, respectively, as determined by comparison of sample NA12878 to reference genotypes provided by the National Institute of Science and Technology. Formal CYP2D6 allele definitions were converted to pseudo-haplotypes (i.e. by a set of discontinuous variants) by reference to the human genome GRCh37.p13. The inheritance of all consensus reference method diplotypes in familial trios and tetrads followed rules of segregation. The analytic sensitivity of the system of the present invention was 96.7% (59 of 61 samples, Table 1). In the remaining two samples the system of the present invention returned more than one diplotype. The analytic specificity of the reference TaqMan® genotype panel and Sanger sequencing were 86.1% (105 of 122 haplotypes) and 83.3% (60 of 72 haplotypes), respectively, while that of the system of the present invention was 89% (105 of 118 haplotypes). The system of the present invention also identified CYP2D6 allelic subtypes (e.g. CYP2D6*1A, *1B, *1D, *1E, *2A, *2M, *3A, *4M, and *4P), albeit these did not alter the prediction of enzymatic activity. In addition, the system of the present invention correctly detected copy number gains (n=2) or losses (n=5) in seven samples. The system of the present invention had two miscalls that that were not shared by the reference methods; it incorrectly identified sample CMH570 as CYP2D6*39/*95 rather than CYP2D6*1/*1, and CMH673 as CYP2D6*83/*35 instead of CYP2D6*1/*35.

The third reference method, quantitative copy number assays, indicated the presence of CYP2D6*68+*4 tandem arrangements in seven individuals. This structure (FIG. 1) cannot be detected by the reference TaqMan® genotype panel, Sanger sequencing, or the system of the present invention. A combination of copy number assays and the system of the present invention had an analytic specificity of 94.9%, which is a fairer comparison with the integrated reference methods.

One advantage of using WGS is that it can identify novel, potentially functionally relevant variation. 15 nucleotide variants were identified by WGS and Sanger Sequencing which are not part of currently defined CYP2D6 alleles (Table 1, FIG. 5). These SNPs define five subvariants of CYP2D6*1 (var1-5), two subvariants of CYP2D6*2 (var1, 2), four subvariants of CYP2D6*4 (var1-4), and one subvariant of CYP2D6*17 (var1).

Below, Table 3 provides novel nucleotide variants identified by WGS. SIFT scores <0.05 are likely deleterious. PolyPhen scores >0.5 are possibly/probably damaging. BLOSUM scores <0 are potentially damaging.

TABLE 3

| chr | start | stop | type | ref | variant | Sanger | hgvs_c |
|---|---|---|---|---|---|---|---|
| 22 | 42522550 | 42522550 | Substitution | G | A | 1 | NM_000106.5: c.*26C > *T; NM_001025161.2: c.#26C > T |
| 22 | 42523241 | 42523241 | Substitution | G | A | | NM_000106.5: c.1173 + 208C > T; NM_001025161.2: c.1020 + 208C > T |
| 22 | 42523309 | 42523309 | Substitution | C | T | | NM_001025161.2: c.1020 + 140G > A; NM_000106.5: c.1173 + 140G > A |
| 22 | 42523315 | 42523315 | Substitution | T | C | | NM_001025161.2: c.1020 + 134A > G; NM_000106.5: c.1173 + 134A > G |
| 22 | 42523400 | 42523400 | Substitution | A | G | | NM_000106.5: c.1173 + 49T > C; NM_0010251612: c.1020 + 49T > C |
| 22 | 42523528 | 42523528 | Substitution | C | T | | NM_001025161.2: c.941G > A; NM_000106.5: c.1094G > A |
| 22 | 42523558 | 42523558 | Substitution | T | C | | NM_001025161.2: c.911A > G; NM_000106.5: c.1064a > G |
| 22 | 42523636 | 42523636 | Substitution | C | A | | NM_0001065: c.986G > T; NM_0010251612: c.833G > T |
| 22 | 42523813 | 42523813 | Substitution | G | A | | NM_001025161.2: c.832 + 31C > T; NM_000106.5: c.985 + 31C > T |
| 22 | 42524033 | 42524033 | Substitution | A | T | 1 | NM_001025161.2: c.691 − 48T > A; NM_000106.5: c.844 − 48T > A |
| 22 | 42524138 | 42524138 | Substitution | C | T | | NM_001025161.2: c.690 + 38G > A; NM_0001065: c.843 + 38G > A |
| 22 | 42524149 | 42524150 | Substitution | — | C | 1 | NM_000106.5: c.843 + 26dupG; NM_001025161.2: c.690 + 26dupG |
| 22 | 42524191 | 42524191 | Substitution | C | A | 1 | NM_001025161.2: c.675G > T; NM_000106.5: c.828G > T |
| 22 | 42524408 | 42524408 | Substitution | A | C | | NM_000106.5: c.667 − 56T > G; NM_001025161.2: c.514 − 56T > G |
| 22 | 42524435 | 42524435 | Substitution | T | A | | NM_000106.5: c.667 − 83A > T; NM_001025161.2: c.514 − 83A > T |
| 22 | 42524708 | 42524708 | Substitution | T | C | | NM_000106.5: c.666 + 78A > G; NM_001025161.2: c.513 + 78A > G |
| 22 | 42524713 | 42524713 | Substitution | C | G | | NM_000106.5: c.666 + 73GC; NM_001025161.2: c.513 + 73G > C |
| 22 | 42524743 | 42524743 | Substitution | G | A | | NM_001025161.2: c.513 + 43C > T; NM_000106.5: c.666 + 43C > T |
| 22 | 42524795 | 42524795 | Substitution | A | G | | NM_001025161.2: c.504T > C; NM_000106.5: c.657T > C |
| 22 | 42524975 | 42524975 | Substitution | C | T | | NM_000106.5: c.506 − 29G > A; NM_001025161.2: c.353 − 29G > A |
| 22 | 42524982 | 42524982 | Substitution | C | T | | NM_000106.5: c.506 − 36G > A; NM_001025161.2: c.353 − 36G > A |
| 22 | 42525039 | 42525039 | Substitution | G | T | | NM_000106.5: c.501C > A; NM_001025161.2: c.353 − 93C > A |
| 22 | 42525239 | 42525239 | Substitution | A | C | 1 | NM_000106.5: c353 − 52T > G; NM_001025161.2: c.353 − 293T > G |
| 22 | 42525534 | 42525534 | Substitution | C | T | 1 | NM_000106.5: c.352 + 206G > A; NM_001025161.2: c.352 + 206G > A |
| 22 | 42525616 | 42525616 | Substitution | C | G | 1 | NM_000106.5: c.352 + 124G > C; NM_001025161.2: c.352 + 124G > C |
| 22 | 42525625 | 42525625 | Substitution | C | T | | NM_001025161.2: c.352 + 115G > A; NM_000106.5: c.352 + 115G > A |
| 22 | 42525645 | 42525645 | Substitution | G | C | 1 | NM_001025161.2: c.352 + 95C > G; NM_000106.5: c.352 + 95C > G |
| 22 | 42525728 | 42525728 | Substitution | A | C | 1 | NM_000106.5: c.352 + 12T > G; NM_001025161.2: c.352 + 12T > G |
| 22 | 42525796 | 42525796 | Substitution | G | T | | NM_000106.5: c.296C > A; NM_001025161.2: c.296C > A |
| 22 | 42525821 | 42525821 | Substitution | G | T | | NM_000106.5: c.271C > A; NM_001025161.2: c.271C > A |
| 22 | 42526370 | 42526370 | Substitution | G | A | 1 | NM_000106.5: c.180 + 244C > T; NM_001025161.2: c.180 + 244C > T |
| 22 | 42526524 | 42526524 | Substitution | G | A | 1 | NM_000106.5: c.180 + 90C > T; NM_001025161.2: c.180 + 90C > T |
| 22 | 42526566 | 42526567 | insertion | — | A | | NM_000106.5: c.180 + 48insT; NM_001025161.2: c.180 + 47_180 + 48insT |
| 22 | 42526571 | 42526571 | Substitution | C | G | | NM_000106.5: c.180 + 43G > C; NM_001025161.2: c.180 + 43G > C |
| 22 | 42526571 | 42526571 | Substitution | C | A | | NM_000106.5: c.180 + 43G > T; NM_001025161.2: c.180 + 43G > T |
| 22 | 42526836 | 42526837 | insertion | — | C | 1 | NM_001025161.2: c.*44dupG; NM_000106.5: c.*44dupG |
| 22 | 42526931 | 42526931 | Substitution | T | C | | |
| 22 | 42527116 | 42527116 | Substitution | A | G | | |
| 22 | 42527191 | 42527191 | Substitution | C | G | | |

| chr | AA change | BLOSUM | SIFT | Polyphen | impact | rsID |
|---|---|---|---|---|---|---|
| 22 | | | | | | rs201759814 |
| 22 | | | | | | |
| 22 | | | | | | |
| 22 | | | | | | |
| 22 | | | | | | rs28578778 |
| 22 | R365H; R314H | 0 | 0 | 0.957 | non_synonymous | rs1058172 |
| 22 | Y355C; Y304C | −2 | 0 | 0.75 | non_synonymous | rs202102799 |
| 22 | R329L; R278L | −2 | 0.12 | 0.728 | non_synonomous | rs3915951 |
| 22 | | | | | | rs143276168 |
| 22 | | | | | | rs28371723 |
| 22 | | | | | | rs372521768 |
| 22 | | | | | | |
| 22 | | | | | | rs28371719 |
| 22 | | | | | | |
| 22 | | | | | | |
| 22 | | | | | | rs111564371 |
| 22 | | | | | | rs112568578 |
| 22 | | | | | | rs113889384 |
| 22 | | | | | | |
| 22 | | | | | | rs200720666 |
| 22 | | | | | | rs113678157 |
| 22 | H167Q | 0 | 0.87 | 0.0002 | non_synonymous | rs1135825 |
| 22 | | | | | | |
| 22 | | | | | | rs267608278 |
| 22 | | | | | | rs180847475 |
| 22 | | | | | | rs1081004 |
| 22 | | | | | | rs186133763 |
| 22 | | | | | | rs78854695 |
| 22 | A99D | −2 | 0 | 0.892 | non_synonymous | |
| 22 | L91M | 2 | 0.01 | 0.973 | non_synonymous | rs28371703 |
| 22 | | | | | | rs267608274 |
| 22 | | | | | | rs29001678 |
| 22 | | | | | | |
| 22 | | | | | | rs74644586 |
| 22 | | | | | | |
| 22 | | | | | | rs75085559 |
| 22 | | | | | | rs267608272 |
| 22 | | | | | | |
| 22 | | | | | | rs374672076 |

Concordance of CYP2D6 Phenotype Prediction

Assignment of correct activity is critical to transition from raw sequencing output to genome-informed drug guidance and precision medicine. Activity scores were assigned to the diplotypes obtained from each platform (TaqMan® genotyping, Sanger sequencing and the system of the present invention) and compared (Table 1). The activity of some CYP2D6 diplotypes is uncertain (function of one or both alleles is unknown at this time), and so it is not possible to predict activities for all of the experimentally defined diplotypes. The clinical sensitivity of the system of the present invention was 93.4% (an activity score was assigned in 57 of 61 subjects), compared with 98.4% (60 of 61) with the consensus reference methods. The clinical specificity of the system of the present invention was 98.2% (56 of 57 Activity Scores were concordant with the consensus reference). Importantly, all extreme phenotypes, i.e. poor and ultrarapid metabolizers were correctly identified with the system of the present invention (Table 1).

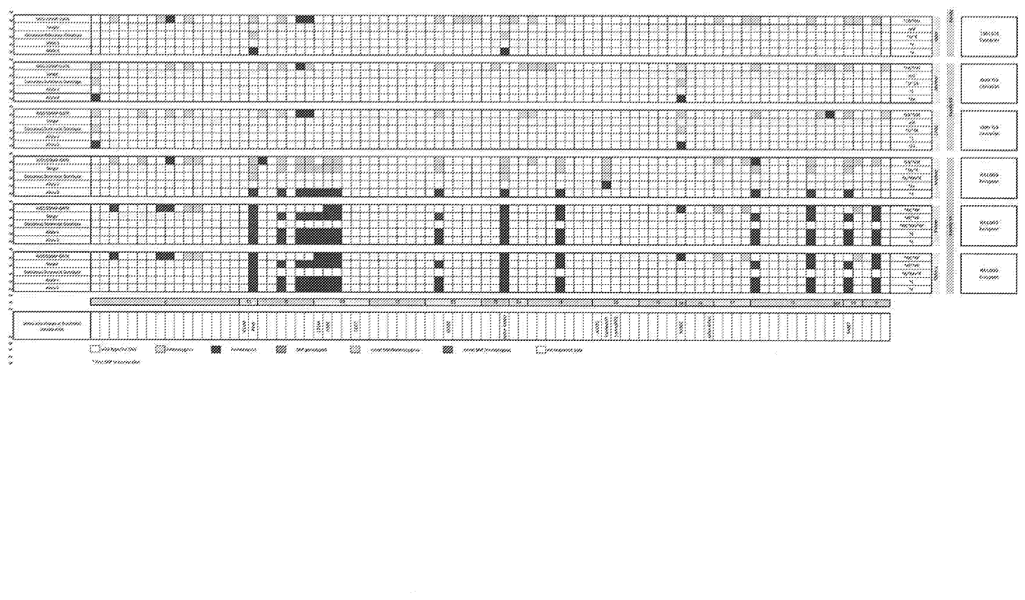

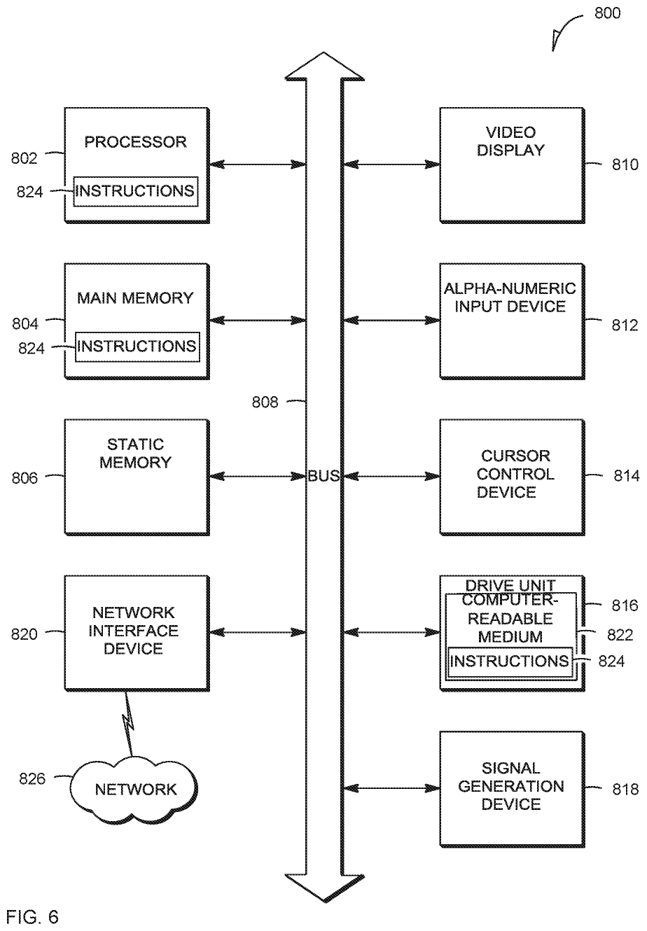

FIG. 6 is a block diagram of an example embodiment of a computer system 800 upon which embodiments of the inventive subject matter can execute. The description of FIG. 6 is intended to provide a brief, general description of suitable computer hardware and a suitable computing environment in conjunction with which the invention may be implemented. In some embodiments, the inventive subject matter is described in the general context of computer-executable instructions, such as program modules, being executed by a computer. Generally, program modules include routines, programs, objects, components, data structures, etc., that perform particular tasks or implement particular abstract data types.

The system as disclosed herein can be spread across many physical hosts. Therefore, many systems and sub-systems of FIG. 6 can be involved in implementing the inventive subject matter disclosed herein. Moreover, those skilled in the art will appreciate that the invention may be practiced with other computer system configurations, including hand-held devices, multiprocessor systems, microprocessor-based or programmable consumer electronics, smart phones, network PCs, minicomputers, mainframe computers, and the like. Embodiments of the invention may also be practiced in distributed computer environments where tasks are performed by I/O remote processing devices that are linked through a communications network. In a distributed computing environment, program modules may be located in both local and remote memory storage devices. Accordingly, it will be appreciated that systems and subsystems may be employed which use cloud-based computing, non-cloud-based computer, and combinations thereof.

In particular, information stored in a computer-readable medium, including without limitation reports generated in accordance with the present invention(s) may be accessed using a variety of types of user-interface access devices, such as mobile communications devices, tablet computers, laptop and desk top computers, etc., having communications functionality for display of such information/reports on a display screen and/or audible output. Additionally, it will be appreciated that systems may include one or more printers and information/reports may be printed and physically distributed or transmitted by electronic communications programs, such as by electronic mail.

With reference to FIG. 6, an example embodiment extends to a machine in the example form of a computer system 800 within which instructions for causing the machine to perform any one or more of the methodologies discussed herein may be executed. In alternative example embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. Further, while only a single machine is illustrated, the term “machine” shall also be taken to include any collection of machines that individually or jointly execute a set (or multiple sets) of instructions to perform any one or more of the methodologies discussed herein.

The example computer system 800 may include a processor 802 (e.g., a central processing unit (CPU), a graphics processing unit (GPU) or both), a main memory 804 and a static memory 806, which communicate with each other via a bus 808. The computer system 800 may further include a video display unit 810 (e.g., a liquid crystal display (LCD) or a cathode ray tube (CRT)). In example embodiments, the computer system 800 also includes one or more of an alpha-numeric input device 812 (e.g., a keyboard), a user interface (UI) navigation device or cursor control device 814 (e.g., a mouse), a disk drive unit 816, a signal generation device 818 (e.g., a speaker), and a network interface device 820.

The disk drive unit 816 includes a machine-readable medium 822 on which is stored one or more sets of instructions 824 and data structures (e.g., software instructions) embodying or used by any one or more of the methodologies or functions described herein. The instructions 824 may also reside, completely or at least partially, within the main memory 804 or within the processor 802 during execution thereof by the computer system 800, the main memory 804 and the processor 802 also constituting machine-readable media.

While the machine-readable medium 822 is shown in an example embodiment to be a single medium, the term “machine-readable medium” may include a single medium or multiple media (e.g., a centralized or distributed database, or associated caches and servers) that store the one or more instructions. The term “machine-readable medium” shall also be taken to include any tangible medium that is capable of storing, encoding, or carrying instructions for execution by the machine and that cause the machine to perform any one or more of the methodologies of embodiments of the present invention, or that is capable of storing, encoding, or carrying data structures used by or associated with such instructions. The term “machine-readable storage medium” shall accordingly be taken to include, but not be limited to, solid-state memories and optical and magnetic media that can store information in a non-transitory manner, i.e., media that is able to store information. Specific examples of machine-readable media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks. The instructions 824 may further be transmitted or received over a communications network 826 using a signal transmission medium via the network interface device 820 and utilizing any one of a number of well-known transfer protocols (e.g., FTP, HTTP). Examples of communication networks include a local area network (LAN), a wide area network (WAN), the Internet, mobile telephone networks, Plain Old Telephone (POTS) networks, and wireless data networks (e.g., WiFi and WiMax networks). The term “machine-readable signal medium” shall be taken to include any transitory intangible medium that is capable of storing, encoding, or carrying instructions for execution by the machine, and includes digital or analog communications signals or other intangible medium to facilitate communication of such software.

From the foregoing it will be seen that this invention is one well adapted to attain all ends and objects hereinabove set forth together with the other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative, and not in a limiting sense.

What is claimed is:

1. A non-transitory computer-readable medium for predicting the diplotype of an individual having computer-executable instructions that when executed causes one or more processors to perform the steps of:
(a) initializing a data store with a plurality of pre-defined locus positions and a plurality of pre-defined nomenclatures;
(b) retrieving genomic sequencing results of an individual;
(c) comparing a plurality of variant calls and associated zygosities with the plurality of pre-defined locus positions and plurality of pre-defined nomenclatures to identify the individual's diplotype;
(d) assigning a score to each of the plurality of pre-defined locus positions based on the comparison of step (c); and
(e) reporting at least one score and associated diplotype to an end user.

2. The computer-readable medium of claim 1 wherein the plurality of pre-defined locus positions consist of a set of genomic locations according to a human genome build against which variants are detected.

3. The computer-readable medium of claim 1 wherein the plurality of pre-defined nomenclatures contains a full set of alleles composed of a plurality of annotated variants.

4. The computer-readable medium of claim 1 wherein the plurality of pre-defined locus positions are a position file that comprises a location of the gene transcript and is located in the data store.

5. The computer-readable medium of claim 1 wherein the plurality of pre-defined nomenclatures comprises a set of possible haplotypes and is located in the data store.

6. The computer-readable medium of claim 1 wherein the step of retrieving the genomic sequencing results of an individual is selected from the steps of whole genome sequencing or next generation sequencing.

7. The computer-readable medium of claim 1 wherein the most likely diplotypes are returned for each plurality of pre-defined nomenclatures.

8. The computer-readable medium of claim 1 wherein the at least one score reported is the highest score from the comparison of step (c) of claim 1.

9. The computer-readable medium of claim 1 further comprising of step (f) predicting biological impact or phenotype of the individual.

10. A non-transitory computer-readable medium for predicting biological impact or phenotype of an individual for use by a medical care provider when selecting medical drugs and assigning an appropriate dosage of the medical drug to the individual having computer-executable instructions that when executed causes one or more processors to perform the step of using an automated identification of genomic variation in genes to determine a diplotype of an individual using the individual's genomic sequence data.

11. A non-transitory computer-readable medium of claim 10 wherein the genomic sequence information is phased genomic sequence information or unphased genomic sequence information.

12. The non-transitory computer-readable medium of claim 10 wherein the gene relates to drug absorption, distribution, metabolism, exertion and response in mammals.

13. A non-transitory computer-readable medium of claim 10 wherein the gene is cytochrome P450 family 2, subfamily D, polypeptide 6.

14. A non-transitory computer-readable medium for predicting a diplotype of an individual for use by a medical care provider having computer-executable instructions that when executed causes one or more processors to perform the steps of:
(a) using a probabilistic scoring system to impute a plurality of diplotypes from genomic sequence data of an individual, wherein the probabilistic scoring system computes a score as the noise corrected likelihood that the genomic sequence data matches a particular diplotype;
(b) assigning to the individual the particular diplotype with the maximum score; and
(c) reporting the particular diplotype with the maximum score to a medical care provider of the individual.
